(19) United States
(12) Patent Application Publication
(10) Pub. No.: US 2005/0032202 A1
(43) Pub. Date: Feb. 10, 2005

(54) Device and method useable for integrated sequential separation and enrichment of proteins

(76) Inventors: Thomas Laurell, Lund (SE); Johan Nilsson, Bjarred (SE); Gyorgy Marko-Varga, Malmo (SE)

Correspondence Address: LADAS & PARRY LLP, 224 SOUTH MICHIGAN AVENUE, SUITE 1200, CHICAGO, IL, 60604, US

(21) Appl. No.: 10/498,194

(22) PCT Filed: Dec. 11, 2002
(86) PCT No.: PCT/SE02/02283

(30) Foreign Application Priority Data
Dec. 11, 2001 (SE) 0104125-0
Jul. 15, 2002 (SE) 0202224-2
Aug. 13, 2002 (SE) 0202399-2

(51) Int. Cl. C12M 1/34

(52) U.S. Cl. 435/287.2; 204/450

(57) ABSTRACT

A device for conducting integrated sequential separation and enrichment of a mixture of sample proteins, comprising means for separation and means for enrichment, whereby free flow electrophoresis—isoelectric focussing (FFE-IEF), is arranged to take place within an etched system of basins and channels is preferably employed in the separation step, multichannel conduits are used to guide the separated protein fractions to the enrichment stage, and a solid phase micro-extraction procedure, in an optionally dockable format, is preferably employed in the enrichment step. The separated fractions are then preferably dispensed onto a MALDI-plate for subsequent matrix assisted laser desorption ionisation (MALDI) analysis.

### FIELD OF INVENTION

The present invention relates to methods and devices for separation, enrichment and analysis of biopolymers. More specifically, it relates to a device for integrated sequential separation and enrichment of proteins.

### BACKGROUND

The identification of new biological targets of medical relevance, aided by human genome research, is an expanding area of modem drug research. These targets may, for example, be receptors responsible for triggering particular responses in the body. While on one hand, attention has focussed on designing and synthesizing potential drug molecules that may interact with these targets, and thus block, reduce or even enhance these responses, the task of identifying the target proteins and target protein complexes themselves has also demanded attention and required improvements.

There is a need for methods allowing rapid and efficient identification of useful peptides, as well as for selecting and identifying relevant peptides, polypeptides and proteins present in a complex biological sample. Such methods exist, but many of these have proven to be slow and labour intensive. In addition, these methods do not make efficient use of the sample as they consume relatively large amounts of test material and are limited in their screening efficiency.

The methods for peptide and protein characterization involving separation and identification have remained largely unchanged for decades. Two of the most popular include Sodium dodecyl sulfate-polyacrylamide-gel electrophoresis (SDS-PAGE) and capillary electrophoresis (CE) see for example Westermeier, R. “Eletrophoresis in Practice: A Guide to methods and Applications of DNA and Protein Separations”, 3^{rd} Ed., Wiley-VCH, Weinheim. Another method is the isoelectric focusing method, see for example “Isoelectric Focusing, principles & methods” published by Pharmacia Fine Chemicals.

Recent prior art is disclosed in a number of patent documents:

WO 00/46594 describes a method, device, kit and system for characterizing proteins based on a variation of the capillary electrophoresis principle, where polypeptides are differentiated by virtue of their molecular weight through electrophoretic migration of the polypeptides through a polymer separation matrix that is contained within a capillary channel. The separation matrix in this invention comprises a polymer matrix, a buffering agent, a detergent and a lipophilic dye. The protein or polypeptide sample to be analyzed is first pretreated with a detergent containing buffer to denature the protein before separation. The treated sample is then introduced into the capillary channel, where an electric field is applied across the length of the channel. The polypeptides, which have been coated with detergent that has substantial charge associated with it, will migrate through the capillary channel.

Polypeptides of different sizes or molecular weight will migrate through the polymer solution or matrix at different rates due to different charge/mass ratios and be separated out. During the migration, the polypeptides pick up the lipophilic dye, making detection possible. An important advantage claimed in this invention include the need for significantly smaller volumes of detergent (for eg., SDS) compared with traditional SDS-PAGE, which reduces dye bonding to detergent micelles, thus decreasing background noise and enhancing detection ability.

WO 99/22228 describes a modular multiple lane micropreparative fraction collection system that permits automated parallel separation and comprehensive collection of all fractions from samples, for example, DNA fragments, in all lanes or columns, with the option of further on-line automated sample analysis of sample fractions. The separation may be carried out using several alternative methods including capillary electrophoresis, capillary isoelectric focusing and capillary electrochromatography, while the detection stage may employ for example, alternative optical methods including laser induced fluorescence, light absorption (UV, visible or IT) using on-column or on-lane detection.

### SUMMARY OF THE INVENTION

The present invention relates to methods and devices for separation, enrichment and analysis of molecules. More specifically, it relates to a device for integrated sequential separation and enrichment of biomolecules e.g. proteins.

A preferred version of a device according to the invention comprises a plate with a length of a few millimetres having an arranged system of basins and channels, and being provided with a cover plate for sealing said channels and basins. The channels and basins are provided with means for performing free flow isoelectric focussing, means for performing solid phase extraction, and means for dispensing a fluid, in a way that a sample solution introduced in the plate is separated into fractions, each fraction then subjected to extraction of relevant molecules, the extracted molecules then enriched and dispensed, thereafter leaving the plate. The solutions are preferably dispensed onto a MALDI-plate for subsequent matrix assisted laser desorption ionisation (MALDI) analysis.

The plate format of the device allows for miniaturisation and integration of a number of parallel flow paths into a single disposable plate or chip.

Although both WO 00/46594 and WO 99/22228 also relate to methods and devices allowing improved separation and analysis of peptides and proteins, neither possesses an extra stage, found in the present invention, for the on-line concentration and enrichment of proteins which allows the detection ability to be enhanced. This enrichment stage is a two step process comprising a solid phase micro-extraction procedure on a porous bed, located in the separation conduits immediately after the separation step followed by dispensing the sample to a small area target where evaporation enriches the sample further. I.e., the proteins enriched on the solid phase bed are subsequently eluted from the porous bed and are transferred as microdroplets to a receiving target plate by means of micro dispensing, e.g., via a piezoelectric microdevice which, similar to ink jet printing, ejects a series of droplets of the enriched and eluted protein sample, in a total volume in the microlitre to nanolitre range.

Also, most importantly the dispensed sample droplets are enriched in this step as they are enriched by rapid solvent evaporation due to the arranged micro-format during the dispensing process, whereby the analyte density on the dried sample spot sequentially is increased for each droplet deposited.

The first step of this two step process enrichment stage may, as an additional feature, be performed in a dockable unit. In an embodiment according to the present invention, the separation stage involves free-flow electrophoresis (FFE), comprising a liquid-based isoelectric focussing (IEF) method found to be a powerful method for resolving proteins. Also, the design of the separation conduits allows for subsequent parallel handling of the separated samples, in, for example, an array format.

### FIGURES

The invention will be described below with the aid of the enclosed figures of which

FIG. 1 shows a first embodiment of a device according to the invention

FIG. 2 shows a second embodiment of a device according to the invention

FIG. 3 shows a block diagram of different embodiments of the invention.

FIG. 4 shows a principal diagram of a two step FFE device.

### DETAILED DESCRIPTION OF PREFERRED EMBODIMENTS

In the following description the term “virtual flow channel” is intended to mean a microscopic flowing portion of a laminary flowing fluid, said portion having a long axis being parallell to the direction of flow, and said portion having a width and a depth orthogonally to the direction of flow, said portion can be regarded as an entity not mixing with the rest of the flowing fluid because said laminar flow and small (micro) dimensions, thus constituting a “virtual channel”. Alternative term: “virtual channel flow”, “virtual flow line” and “virtual flow lane”.

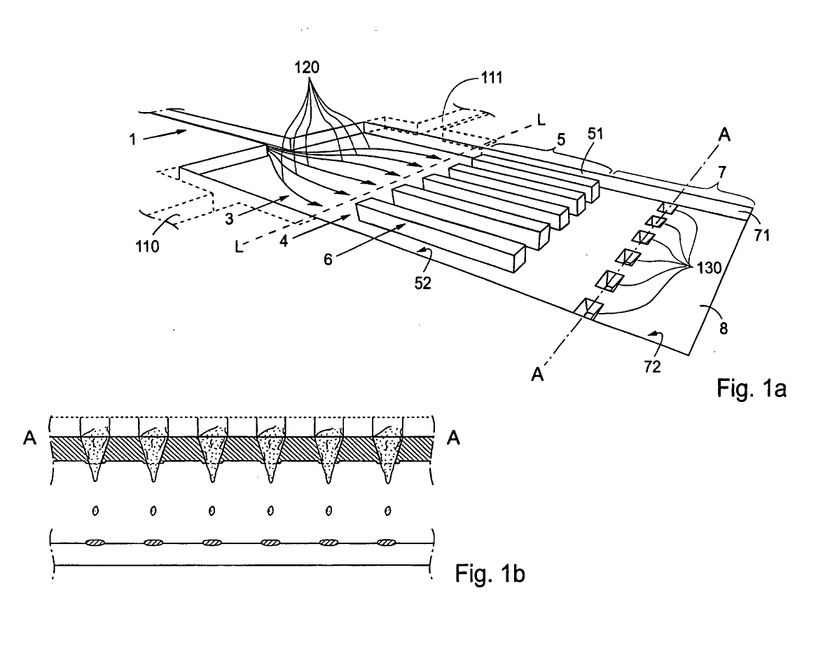

As can be derived from FIG. 1 one of the embodiments of the invention relates to a device for integrated sequential gel-free separation and enrichment of proteins. Also, a method for separating and enriching said proteins by the use of said device is contemplated. Said separation and enrichment method(s) and device(s) are convenient combinations of known principles and methods as well as new methods and devices. The combination will give improved effects on efficiency for a device according to the invention in respect of sample handling time, due to less laborious manual input, as well as protein resolution in the identification step.

The device for conducting integrated sequential separation and enrichment of proteins in a mixture of protein molecules and solvents is schematically outlined in FIG. 1. The device comprises means for separation and means for enrichment.

Via a sample inlet 2 and buffer inlet 1, which optionally may be the same, the samples are delivered to the separation portion 31, separating the sample proteins orthogonal to the sample buffer flow. Said separation portion 31 comprises a separation basin 3 and means for separation arranged in fluid communication with said inlets 1, 2 at a small plate and having a distance from a focus line (L) of the separation means, thought as an imaginary line between the most downstream part of a pair of electrodes 110, 111, to a front line (F) of the extraction means (corresponding to the upstream start of the separating walls), which distance is small enough to prevent significant diffusion of biomolecules from one separated fraction/laminar-flow portion to another through a pattern of laminar flow. After the separation, conduits 4 lead the separated samples to a second portion 5, a micro-extraction portion comprising micro-extraction means, of the device suitable for enrichment of the proteins. The portion 5 comprises micro-extraction means (not shown) and separating walls 6. The micro-extraction portion 5 may optionally be a dockable unit. With this term is meant that said portion comprises a unit attachable to, detachable from, and reattachable to other devices/units.

The device further comprises a dispensing portion 7 comprising a dispenser basin 8 with a number of nozzle openings 130, said portion 7 being arranged in fluid communication with said extraction means such that a defining surface 71, 72 of the dispenser basin comprises the elongation of the outer surfaces 51, 52 of the extraction means. Said basin 8 is devised without dividing walls to keep dimensions as small as possible. The problem that diffusion would mix the separated portions is solved by the speed of the flow, i.e., there is not enough time for the flow to laterally mix by diffusion before it is dispensed or flowed past the nozzle openings 130 due to the governing laminar flow conditions.

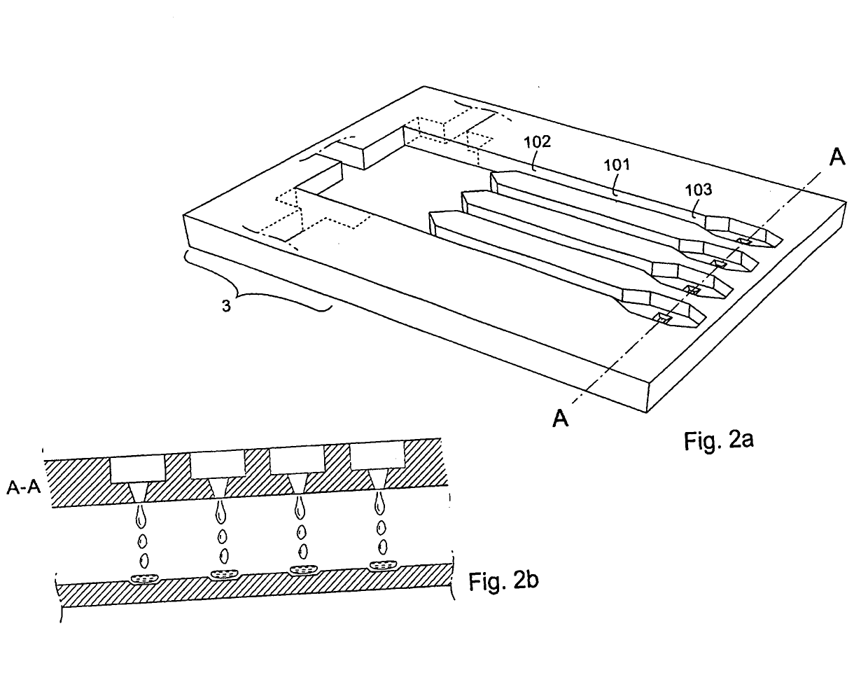

FIG. 2 shows a second embodiment of the device comprising an alternative design which comprises separating walls, separating the flows all the way to the dispenser nozzle opening.

The above described portions comprises in alternative combinations separate units, dockable to each other. E.g. a separation unit comprising docking means and separation means in the shape of free flow electrophoresis means can be docked to an enrichment unit comprising docking means and solid phase enrichment means. Said docking means comprises connections such that sample solutions can be made to flow from one unit to another.

In a method making use of the device, sample proteins are initially separated in a gel-free separation process in a first portion 3 devised therefore, preferably by the use of a free-flow electrophoresis (FFE). The sample proteins/molecules are separated preferably by pH (isoelectric focussing, IEF). At the end of the FFE, when the flowing sample solution has been subjected to FFE, and proteins have become separated into parallely free flowing laminar flows, separated sample proteins are kept apart in separate conduits 4 having separation walls 6, arranged immediately after (in the direction of flow) the FFE. These conduits 4 constitute a second portion 5 that allows for a subsequent parallel handling of the separated samples in e.g. an array-format, by eventually dispensing a number of parallel samples repeatedly to fill an array format plate, e.g. a 96-, 384- or even a higher order well format, or in a convenient strip format in e.g. rows of 12, or more.

A typical step that can be performed in the second portion 5 is an enrichment procedure, preferably a solid phase micro-extraction procedure (SPE) on a porous bed, e.g. by the use of particles. Said SPE can be performed in an integrated microextraction array-chip that can comprise bead particles packed in the chip or can comprise a highly porous silicon or polymer structure with appropriate surface functionality that has a high affinity towards the proteins to be analyzed.

In another embodiment said micro-extraction procedure proceeds in a optionally dockable microextraction unit positioned directly after the separation conduits mentioned above. By eluting/dispensing sample proteins in a specified small volume, i.e. in the microlitre-nanolitre range, the sample elution from the porous bed and integrated on-line fraction collection by means of micro dispensing (“ink-jetting”) and rapid evaporation, sample proteins are enriched in a two step process.

To accomplish the high level of integration that is required for the described protein separation and enrichment procedure the system is preferably fabricated by means of micro- and nanotechnology. The need for miniatyrisation and a compact system integration stems from the fact that the original protein sample may be very dilute or the volume extremely small, which thus requires a minimum of sample handling steps in the analysis procedure to avoid loss of analyte molecules when performing the bioanalytical protocol.

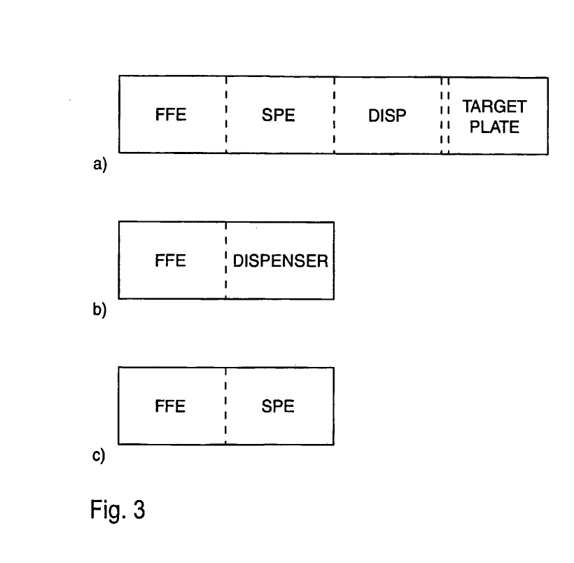

FIG. 3 shows 3 other embodiments of the device where the separation, microextraction, dispensing and target analysis stages are all in the form of dockable units which can be assembled in various alternative ways.

Two step FFE

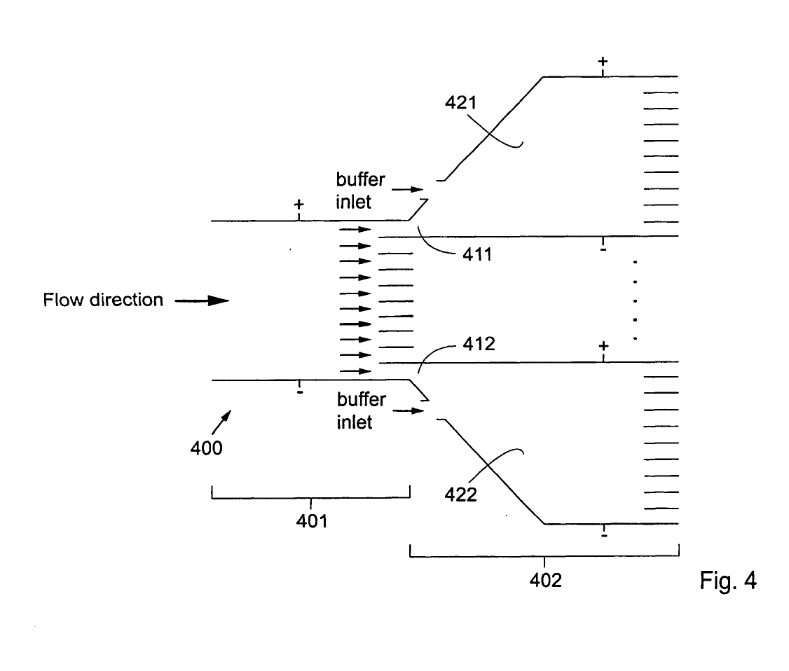

In a two step device 400 according to an embodiment of the invention, shown in FIG. 4, FFE portions/chips are arranged so that the resulting sample solutions from a first separation portion/chip 401 is fed to a second separation step 402, comprising a multitude of separation chips 421, 422 etc, having fluid connection 411, 412 etc with said first step chip 401, and provided with appropriate ampholytic buffers and applied voltages such that a separation into more fluid portions can be achieved. A typical embodiment may separate an incoming sample solution into ten fluid portions in a first step 401 and then each of those portions into ten subportions, making a total separation into one hundred fluid portions. Each of these fluid portions is then advantageously subjected to array dispensing on one or more MALDI target plates for subsequent MALDI analysis.

1. A device useable for separation and enrichment of a mixture of sample molecules in solution, characterised in that said device comprises means for separation (31) and means for enrichment, arranged in fluid communication at a small plate and having a distance from a focus line (L) of the separation means to a front line (F) of the extraction means small enough to prevent significant diffusion of biomolecules from one laminar flow portion to another.

2. The device according to claim 1, wherein said means for separation (31) comprises an inlet (1), a basin (3) and electrodes (110, 111) arranged at sides of said basin (3) such that a fluid flowing from the inlet (1) can be separated into fractions (120) containing biomolecules having different isoelectric points by means of free flow isoelectric focussing.

3. The device according to claim 2, wherein said electrodes are arranged within side compartments of the basin (3) having fluid connection to said basin (3).

4. The device according to claim 1, wherein said means for extraction (5) comprises two outer surfaces (51, 52) and a number of separating walls, separating a number of channels, each channel being provided with adsorption means.

5. The device according to claim 1, further comprising a dispensing zone (7) comprising a dispenser basin (8) with a number of nozzle openings (130), said zone being arranged in fluid communication with said extraction means such that a defining surface (71, 72) of the dispenser basin comprises the elongation of the outer surfaces (51, 52) of the extraction means.

6. A solid phase extraction unit comprising means for enrichment and means for docking such that said extraction unit can be docked to another unit, e.g. a dispensing unit.

7. A free flow electrophoresis unit comprising means for free flow electrophoresis and means for docking, such that said electrophoresis unit can be docked to another unit, e.g. a solid phase extraction unit.

8. The device according to claim 1 wherein the separation means is devised to separate proteins in a direction orthogonal relatively to the direction of the flow of the said solution containing said proteins.

9. A method for separation and enrichment of a mixture of sample biomolecules in solution, characterised in that said method comprises the following steps
feeding a flow of a solution to a separation device
feeding a flow of a buffer ampholyte to the same separation device
applying a voltage over the flow, orthogonally to the direction of the flow such that a pH gradient is established in the buffer ampholyte and proteins are made to migrate towards their isoelectric points, thereby separating proteins having different isoelectric points.

10. The method as recited in claim 8 further comprising the steps of
collecting separate fractions of the flow containing said proteins with different isoelectric points.

11. The method as recited in claim 10 further comprising the step of feeding separated proteins to an enrichment device.

12. The method as recited in claim 11, where said enrichment device is a solid phase enrichment device.

13. The method as recited in claim 12, further comprising the steps of depositing each of the SPE enriched portions in a plate by means of microdispensing.

14. The method as recited in claim 13, where said dispensing is performed with an array dispenser.
